(19) United States
(12) Patent Application Publication
(10) Pub. No.: US 2026/0200850 A1
(43) Pub. Date: Jul. 16, 2026

(54) CRYSTALLINE FORMS OF A PIPERIDINE INHIBITOR OF SLC6A19 FUNCTION

(71) Applicant: Jnana Therapeutics Inc., Boston, MA (US)

(72) Inventors: Mitchell T. Antalek, Medford, MA (US); Daniel T. Walters, Cranbury, NJ (US); Maria YOUNG, Cranbury, NJ (US); Aniket S. Puri, Cranbury, NJ (US)

(21) Appl. No.: 19/134,083

(22) PCT Filed: Nov. 29, 2023
(86) PCT No.: PCT/US2023/081526

§ 371 Date: May 29, 2025

Related U.S. Application Data
(60) Provisional application No. 63/428,867, filed on Nov. 30, 2022.

(51) Int. Cl. C07D 211/56

(52) U.S. Cl. CPC C07D 211/56

(57) ABSTRACT

Disclosed are crystalline forms of the compound of formula (1), compositions, and methods useful for treating or preventing a disease or disorder associated with abnormal levels of amino acids by modulation of SLC6A19 transport.

### RELATED APPLICATION

This application claims the benefit of priority to U.S. Provisional Application No. 63/428,867, filed Nov. 30, 2022.

### BACKGROUND

Phenylketonuria (PKU) is an inborn error of metabolism caused by mutations in phenylalanine hydroxylase (PAH), the enzyme responsible for metabolizing phenylalanine. PKU is an autosomal recessive metabolic disorder in which phenylalanine is not properly metabolized and results in abnormally high levels of plasma phenylalanine. People who have PKU have abnormally high blood levels of phenylalanine, which if untreated can lead to irreversible neurological damage resulting in a spectrum of complications such as intellectual disabilities, seizures, neurodevelopmental and behavioral disorders. PKU is difficult to treat because blood levels of phenylalanine are directly related to diet. Patients must adhere to a life-long and strict diet that impacts all aspects of patients' lives. Current standard of care are enzyme co-factor and enzyme substitution therapy but these therapies are not effective in all patients, and carry potential risk for adverse events.

The enzyme responsible for metabolizing phenylalanine, and thus maintaining phenylalanine homeostasis is phenylalanine hydroxylase (PAH). Loss-of-function (LOF) mutations at PAH gene at chromosome 12q23.2 are known to cause most forms of PKU. These LOF mutations resulting in PKU can be diagnosed as classical PKU (the most severe form), and “mild PKU” or “hyperphe” a less severe form. In addition to PAH, mutations in other enyzmes that affect phenylalanine metabolism, such as dihydropteridine reductase (DHPR), the enzyme responsible for synthesis of co-factors required for PAH activity, may also result in elevated levels of phenylalanine. In addition to diet, blood amino acid levels, including levels of phenylalanine, are regulated by SLC6A19. SCL6A19 is located in the proximal tubule of the kidney and is responsible for reabsorption of amino acids back into the blood.

### SUMMARY

One aspect of the invention provides crystalline forms, compositions, and methods useful for treating or preventing a disease or disorder associated with abnormal levels of amino acids by modulation of SLC6A19 transport.

Accordingly, provided herein are crystalline forms of compound (1), having the following absolute stereochemical configuration:

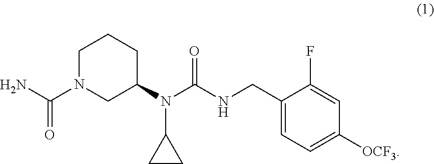 [SMILES: NC(=O)N1CCC[C@@H](N(C(=O)NCc2ccc(OC(F)(F)F)cc2F)C2CC2)C1]

Also provided herein is crystalline Form I of compound (1), having the following absolute stereochemical configuration:

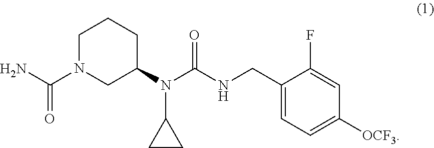 [SMILES: NC(=O)N1CCC[C@@H](N(C(=O)NCc2ccc(OC(F)(F)F)cc2F)C2CC2)C1]

Also provided herein is crystalline Form II of compound (1), having the following absolute stereochemical configuration:

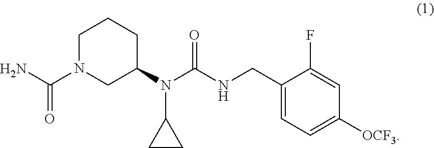 [SMILES: NC(=O)N1CCC[C@@H](N(C(=O)NCc2ccc(OC(F)(F)F)cc2F)C2CC2)C1]

Another aspect of the invention relates to methods of treating or preventing a disease or disorder associated with a genetic defect in phenylalanine hydroxylase in a subject in need thereof comprising administering to the subject an effective amount of a crystalline form of compound (1).

Another aspect of the invention relates to methods of treating or preventing phenylketonuria comprising administering to the subject an effective amount of a crystalline form of compound (1).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is an X-Ray Powder Diffraction (XRPD) pattern of polymorphic Form I.

FIG. 2 is a polarized light microscopy (PLM) image of polymorphic Form I.

FIG. 3 is a Thermogravimetric Analysis (TGA)/Differential Scanning calorimetry (DSC) spectrum of polymorphic Form I.

FIG. 4 is an X-Ray Powder Diffraction (XRPD) pattern of polymorphic Form II.

FIG. 5 is a polarized light microscopy (PLM) image of polymorphic Form II.

FIG. 6 is a Thermogravimetric Analysis (TGA)/Differential Scanning calorimetry (DSC) spectrum of polymorphic Form II.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

For purposes of this invention, the chemical elements are identified in accordance with the Periodic Table of the Elements, CAS version, Handbook of Chemistry and Physics, 67th Ed., 1986-87, inside cover.

### Crystalline Forms

One aspect of the invention provides crystalline form of compound (1), having the following absolute stereochemical configuration:

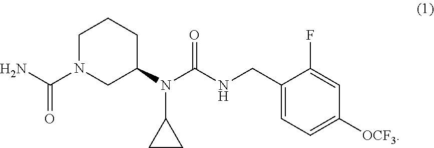 [SMILES: NC(=O)N1CCC[C@@H](N(C(=O)NCc2ccc(OC(F)(F)F)cc2F)C2CC2)C1]

In some embodiments, the crystalline form is anhydrous.

In some embodiments, the crystalline form (Form I) is characterized by at least three X-ray powder diffraction (XRPD) peaks at values of two theta (°2θ±0.2°; or °2θ) selected from 3.60, 10.87, 12.67, 20.91, 21.48, and 21.91.

In some embodiments, the crystalline form is characterized by at least four X-ray powder diffraction (XRPD) peaks at values of two theta (°2θ±0.2°; or °2θ) selected from 3.60, 10.87, 12.67, 20.91, 21.48, and 21.91.

In some embodiments, the crystalline form is characterized by at least five X-ray powder diffraction (XRPD) peaks at values of two theta (°2θ±0.2°; or °2θ) selected from 3.60, 10.87, 12.67, 20.91, 21.48, and 21.91.

In some embodiments, the crystalline form is characterized by X-ray powder diffraction (XRPD) peaks at values of two theta (°2θ±0.2°; or °2θ) of 3.60, 10.87, 12.67, 20.91, 21.48, and 21.91.

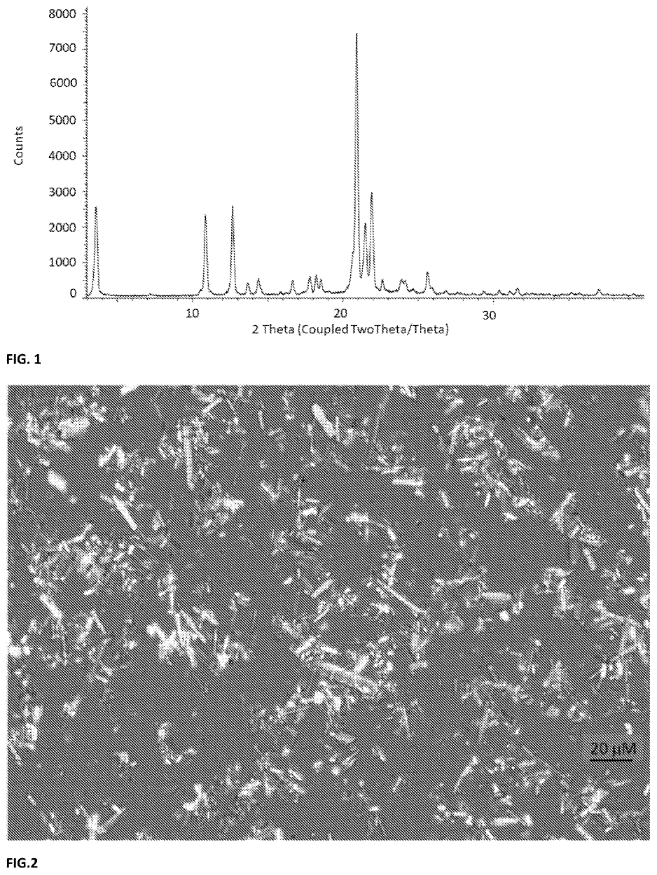

In some embodiments, the crystalline form is characterized by an X-ray powder diffraction (XRPD) pattern substantially in accordance with that depicted in FIG. 1.

In some embodiments, the crystalline form is further characterized by a differential scanning calorimetry (DSC) thermogram comprising an endothermic peak at about 158.1° C.

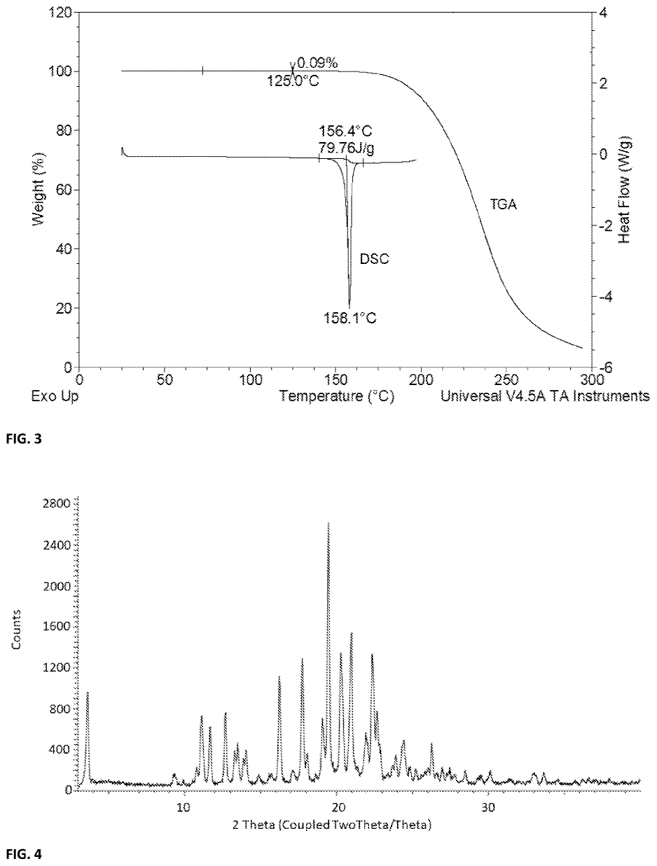

In some embodiments, the crystalline form is further characterized by a DSC thermogram substantially in accordance with the DSC thermogram in FIG. 3.

In some embodiments, the crystalline form is further characterized by a thermal gravimetric analysis (TGA) thermogram substantially in accordance with the TGA thermogram in FIG. 3.

In some embodiments, the crystalline form (Form II) is characterized by at least eight X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

In some embodiments, the crystalline form is characterized by at least nine X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

In some embodiments, the crystalline form is characterized by at least ten X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

In some embodiments, the crystalline form is characterized by at least eleven X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

In some embodiments, the crystalline form is characterized by X-ray powder diffraction (XRPD) peaks at values of two theta (°2θ±0.2°; or °2θ) of 3.61, 11.13, 11.70, 12.70, 16.25, 17.76, 19.09, 19.47, 20.29, 20.97, 22.37, and 22.72.

In some embodiments, the crystalline form (Form II) is characterized by at least three X-ray powder diffraction (XRPD) peaks at values of two theta (°2θ±0.2°; or °2θ) selected from 17.76, 19.47, 20.29, 20.97, and 22.37.

In some embodiments, the crystalline form is characterized by at least four X-ray powder diffraction (XRPD) peaks at values of two theta (°2θ±0.2°; or °2θ) selected from 17.76, 19.47, 20.29, 20.97, and 22.37.

In some embodiments, the crystalline form is characterized by at least five X-ray powder diffraction (XRPD) peaks at values of two theta (°2θ±0.2°; or °2θ) selected from 17.76, 19.47, 20.29, 20.97, and 22.37.

In some embodiments, the crystalline form is characterized by X-ray powder diffraction (XRPD) peaks at values of two theta (°2θ±0.2°; or °2θ) of 17.76, 19.47, 20.29, 20.97, and 22.37.

In some embodiments, the crystalline form is characterized by an X-ray powder diffraction (XRPD) pattern substantially in accordance with that depicted in FIG. 4.

In some embodiments, the crystalline form is further characterized by a DSC thermogram comprising a endothermic peak at about 160.4° C.

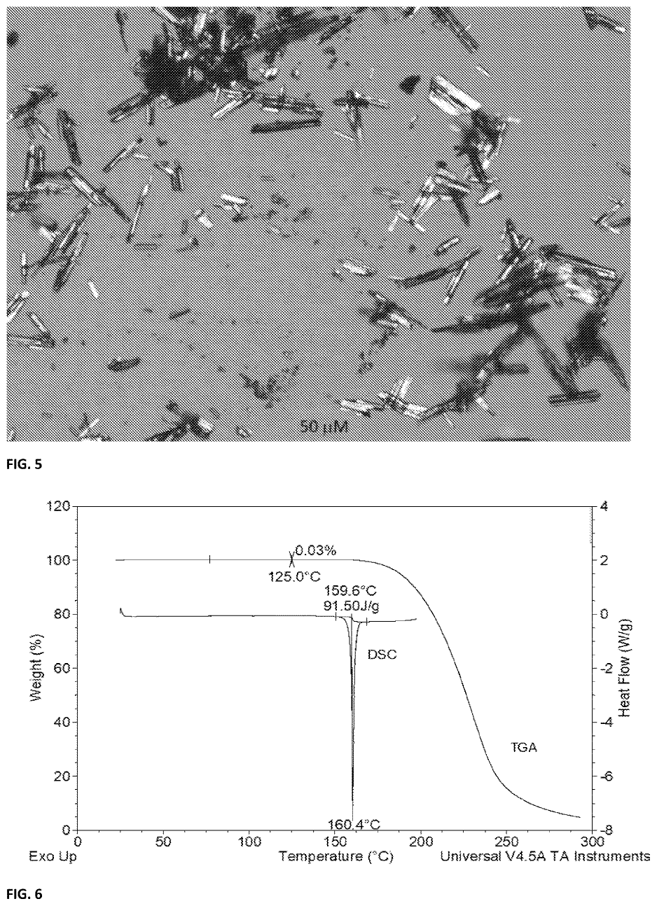

In some embodiments, the crystalline form is further characterized by a DSC thermogram substantially in accordance with the DSC thermogram in FIG. 6.

In some embodiments, the crystalline form is further characterized by a TGA thermogram substantially in accordance with the TGA thermogram in FIG. 6.

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Any compound of the invention can also be radiolabed for the preparation of a radiopharmaceutical agent.

### Methods of Treatment

One aspect of the invention provides crystalline forms, compositions, and methods useful for treating or preventing a disease or disorder associated with abnormal levels of amino acids by modulation of SLC6A19 transport.

Another aspect of the invention provides a method of treating or preventing a disease or disorder associated with a genetic defect in phenylalanine hydroxylase comprising administering to the subject an effective amount of a crystalline form of compound (1).

In some embodiments, the disease or disorder associated with a genetic defect in phenylalanine hydroxylase is phenylketonuria. In some embodiments, the phenylketonuria is classical phenylketonuria. In other eirihodiirients, the phenylketonuria is mild phenylketonuria.

In some embodiments, the disease or disorder associated with a genetic defect in phenylalanine hydroxylase is hyperphenylalaninemia.

In certain embodiments, the compound inhibits SLC6A19 in the patient.

In some embodiments of any one of the disclosed methods, wherein the subject is a mammal. In some embodiments of any one of the disclosed methods, the mammal is a human.

In some embodiments of any one of the disclosed methods, the crystalline form is

### Form I.

In some embodiments of any one of the disclosed methods, the crystalline form is Form II.

### Pharmaceutical Compositions, Routes of Administration, and Dosing

In certain embodiments, the invention is directed to a pharmaceutical composition, comprising a crystalline form, e.g. Form I or Form II, of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, «-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989)(α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly (butylmethacrylate), poly(isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), and poly (octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Abbreviations

DSC=differential scanning calorimetry
FaSSIF=fasted state simulated intestinal fluid
FeSSIF=fed state simulated intestinal fluid
HPBCD=hydroxyl-propyl-beta cyclodextrin
PLM=polarized light microscopy
SGF=simulated gastric intestinal fluid
TGA=thermogravimetric analysis
TPGS=D-alpha-tocopheryl polyethylene glycol 1000 succinate
V=volume

A synthesis of amorphous of (R)-3-(1-cyclopropyl-3-(2-fluoro-4-(trifluoromethoxy)benzyl) ureido) piperidine-1-carboxamide is described in WO 2022/192370 (which is incorporated by reference in its entirety).

### Example 1: Preparation of Polymorphic Form I

(R)-3-(1-cyclopropyl-3-(2-fluoro-4-(trifluoromethoxy)benzyl) ureido) piperidine-1-carboxamide (1.91 kg) was charged into a reactor and methyl tert-butyl ether (MTBE) (19.1 L) was added. The mixture was stirred at 40-50° C. Solid was collected by filtration (wet cake 1.88 kg) and was washed with MTBE (3.8 L) and dried at 40-50° C. to give the material (1.825 kg, 95.5% yield) as the Form I polymorph. Characterization by XRPD is provided in FIG. 1 with a tabulated spectra of XRPD provided in Table 1. A PLM scan is provided in FIG. 2. DSC/TGA measurements are provided in FIG. 3.

### Example 2: Preparation of Polymorphic Form II

Three grams of Form I was dissolved in 9 volumes (27 mL) of methyl ethyl ketone (MEK) at 50° C. The solution was cooled to 40° C. and to this was added 1% of Form II seed crystal and held for one hour. (The seed crystals were prepared by dissolving 200 mg of Form I in MEK at 50° C., and then cooling, and filtration after crystals appeared.) The solution was slowly cooled to 20° C. and then charged with 54 mL of heptane at 1.5V/h. The solids were collected by filtration and washed with 2V of heptane and then dried on a vacuum at 40° C. for 2 hours to give 92.3% yield of Form II with a tabulated spectra of XRPD provided in Table 2. A PLM scan is provided in FIG. 5. DSC/TGA measurements are provided in FIG. 6.

TABLE 1
Tabulated Spectra of XRPD for polymorphic Form I.

| Angle | d Value | Rel. Intensity | Intensity |
|---|---|---|---|
| 3.595 | 24.55872 | 29.30% | 1527.43 |
| 7.221 | 12.23247 | 0.60% | 91.7857 |
| 10.534 | 8.39153 | 1.70% | 165.658 |
| 10.868 | 8.13425 | 29.80% | 1570.06 |
| 12.668 | 6.9821 | 33.20% | 1755.54 |
| 13.684 | 6.46591 | 4.00% | 309.246 |
| 14.383 | 6.15334 | 6.40% | 425.75 |
| 16.662 | 5.31631 | 5.00% | 358.936 |
| 17.798 | 4.97947 | 6.70% | 467.679 |
| 18.212 | 4.86725 | 7.10% | 494.2 |
| 18.533 | 4.78375 | 5.10% | 395.748 |
| 19.027 | 4.6607 | 0.90% | 189.351 |
| 20.913 | 4.24441 | 100.00% | 5156.17 |
| 21.484 | 4.1328 | 25.60% | 1459.05 |
| 21.91 | 4.05334 | 38.00% | 2080.08 |
| 22.627 | 3.92653 | 5.20% | 440.663 |
| 23.941 | 3.7139 | 3.70% | 347.476 |
| 24.144 | 3.6831 | 4.50% | 384.124 |
| 24.645 | 3.60938 | 1.20% | 205.251 |
| 25.622 | 3.47397 | 8.30% | 543.444 |
| 26.843 | 3.31866 | 1.60% | 191.639 |
| 27.611 | 3.22807 | 1.10% | 151.375 |
| 28.631 | 3.1153 | 0.60% | 117.693 |
| 29.373 | 3.03829 | 1.40% | 153.725 |
| 30.404 | 2.93754 | 2.00% | 182.961 |
| 31.115 | 2.87205 | 1.20% | 142.886 |
| 31.597 | 2.82933 | 3.20% | 239.118 |
| 32.206 | 2.77721 | 1.00% | 128.871 |
| 32.581 | 2.74613 | 0.70% | 113.658 |
| 33.167 | 2.6989 | 0.50% | 106.733 |
| 33.699 | 2.65753 | 0.80% | 115.829 |
| 35.199 | 2.5476 | 1.00% | 129.292 |
| 35.689 | 2.51376 | 0.50% | 104.322 |
| 37.011 | 2.42692 | 2.70% | 209.084 |
| 37.577 | 2.3917 | 0.50% | 98.8872 |
| 39.324 | 2.28937 | 0.50% | 87.3634 |

TABLE 2
Tabulated Spectra of XRPD for polymorphic Form II.

| Angle | d Value | Rel. Intensity | Intensity |
|---|---|---|---|
| 3.612 | 24.44512 | 29.40% | 558.685 |
| 9.337 | 9.46392 | 2.70% | 100.78 |
| 9.922 | 8.90711 | 0.80% | 67.8101 |
| 10.544 | 8.38301 | 1.70% | 83.5982 |
| 10.819 | 8.17058 | 5.40% | 152.563 |
| 11.131 | 7.94249 | 26.40% | 515.712 |
| 11.697 | 7.55935 | 21.20% | 431.158 |
| 12.697 | 6.96624 | 25.70% | 515.705 |
| 13.321 | 6.64114 | 11.60% | 276.104 |
| 13.502 | 6.55267 | 14.20% | 321.316 |
| 13.893 | 6.36918 | 8.60% | 223.426 |
| 14.054 | 6.29641 | 12.40% | 287.003 |
| 14.867 | 5.95417 | 3.40% | 132.636 |
| 15.717 | 5.63372 | 3.20% | 136.423 |
| 16.249 | 5.45067 | 39.30% | 759.153 |
| 17.125 | 5.17361 | 4.20% | 162.022 |
| 17.758 | 4.99073 | 46.50% | 894 |
| 18.074 | 4.90407 | 11.00% | 290.557 |
| 18.62 | 4.76144 | 2.90% | 158.275 |
| 19.088 | 4.64573 | 23.00% | 514.511 |
| 19.472 | 4.55501 | 100.00% | 1839.98 |
| 20.292 | 4.37289 | 47.00% | 944.575 |
| 20.97 | 4.23296 | 53.80% | 1065.69 |
| 21.946 | 4.04692 | 17.40% | 442.66 |
| 22.369 | 3.97118 | 47.40% | 951.778 |
| 22.724 | 3.90997 | 20.70% | 490.607 |
| 23.396 | 3.79924 | 2.60% | 172.44 |
| 23.699 | 3.75135 | 4.40% | 203.552 |
| 23.903 | 3.7197 | 9.10% | 282.125 |
| 24.431 | 3.6405 | 15.70% | 389.288 |
| 24.835 | 3.58227 | 3.80% | 179.988 |
| 25.213 | 3.52934 | 4.40% | 188.491 |
| 26.042 | 3.41889 | 4.40% | 183.809 |
| 26.273 | 3.38931 | 15.60% | 373.519 |
| 26.609 | 3.34732 | 2.40% | 144.923 |
| 26.957 | 3.30487 | 4.20% | 170.865 |
| 27.405 | 3.25186 | 4.30% | 168.886 |
| 27.775 | 3.20936 | 3.00% | 141.804 |
| 28.477 | 3.13183 | 5.40% | 169.198 |
| 29.552 | 3.02026 | 3.20% | 126.578 |
| 30.126 | 2.96408 | 4.80% | 155.211 |
| 31.375 | 2.84884 | 1.10% | 88.4157 |
| 32.296 | 2.76971 | 1.10% | 81.7808 |
| 32.979 | 2.71388 | 3.60% | 127.383 |
| 33.643 | 2.66182 | 5.30% | 159.553 |
| 34.575 | 2.59215 | 1.30% | 91.7166 |
| 35.701 | 2.5129 | 1.70% | 91.2006 |
| 36.22 | 2.47812 | 2.00% | 97.8437 |
| 36.605 | 2.45294 | 2.50% | 109.892 |
| 37.088 | 2.4221 | 1.80% | 99.3526 |
| 37.961 | 2.36835 | 2.70% | 115.852 |

### Example 3: Solubility Measurements of Form I and Form II

Solubility measurements of Form I and Form II were conducted in relevant biofluids and are reported in Table 3. Form I has increased solubility relative to Form II.

TABLE 3
Solubility measurements of polymorphic
form I and II in biorelevant fluids

| | Polymorph | | Solubility (mg/mL) | | |
|---|---|---|---|---|---|
| Media | Form | Temperature | 1.5 h | 3 h | 24 h |
| FaSSIF | I | 37° C. | 0.36 | 0.38 | 0.35 |
| | II | | 0.31 | 0.27 | 0.24 |
| FeSSIF | I | 37° C. | 0.91 | 0.90 | 0.98 |
| | II | | 0.74 | 0.72 | 0.72 |
| SGF | I | 37° C. | 0.40 | 0.42 | 0.37 |
| | II | | 0.26 | 0.27 | 0.30 |
| 5% HPBCD/2% TPGS | I | 25° C. | 4.96 | 5.11 | 5.28 |
| | II | | 4.31 | 4.27 | 4.76 |

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A crystalline form of compound (1), having the following absolute stereochemical configuration:

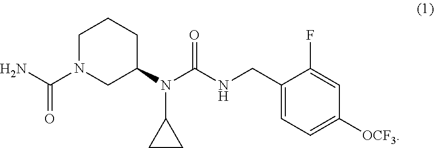 [SMILES: NC(=O)N1CCC[C@@H](N(C(=O)NCc2ccc(OC(F)(F)F)cc2F)C2CC2)C1]

2. The crystalline form of claim 1, wherein the crystalline form is anhydrous.

3. The crystalline form of claim 1 or 2, wherein the crystalline form (Form I) is characterized by at least three X-ray powder diffraction (XRPD) peaks at values of two theta (°2θ±0.2°; or °2θ) selected from 3.60, 10.87, 12.67, 20.91, 21.48, and 21.91.

4. The crystalline form of claim 3, wherein the crystalline form is characterized by at least four X-ray powder diffraction (XRPD) peaks at values of two theta (°2θ±0.2°; or °2θ) selected from 3.60, 10.87, 12.67, 20.91, 21.48, and 21.91.

5. The crystalline form of claim 3 or 4, wherein the crystalline form is characterized by at least five X-ray powder diffraction (XRPD) peaks at values of two theta (°2θ±0.2°; or °2θ) selected from 3.60, 10.87, 12.67, 20.91, 21.48, and 21.91.

6. The crystalline form of any one of claims 3-5, wherein the crystalline form is characterized by X-ray powder diffraction (XRPD) peaks at values of two theta (°2θ±0.2°; or °2θ) of 3.60, 10.87, 12.67, 20.91, 21.48, and 21.91.

7. The crystalline form of any of claims 1-6, wherein the crystalline form is characterized by an X-ray powder diffraction (XRPD) pattern substantially in accordance with that depicted in FIG. 1.

8. The crystalline form of any one of claims 1-7, wherein the crystalline form is further characterized by a differential scanning calorimetry (DSC) thermogram comprising a endothermic peak at about 158.1° C.

9. The crystalline form of any one of claims 1-8, wherein the crystalline form is further characterized by a DSC thermogram substantially in accordance with the DSC thermogram in FIG. 3.

10. The crystalline form of any one of claims 1-9, wherein the crystalline form is further characterized by a thermal gravimetric analysis (TGA) thermogram substantially in accordance with the TGA thermogram in FIG. 3.

11. The crystalline form of claim 1 or 2, wherein the crystalline form (Form II) is characterized by at least eight X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

12. The crystalline form of claim 11, wherein the crystalline form is characterized by at least nine X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

13. The crystalline form of claim 11 or 12, wherein the crystalline form is characterized by at least ten X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

14. The crystalline form of any one of claims 11-13, wherein the crystalline form is characterized by at least eleven X-ray powder diffraction (XRPD) peaks at values of two theta (°2θ±0.2°; or °2θ) selected from 3.61, 11.13, 11.70, 12.70, 16.25, 17.76, 19.09, 19.47, 20.29, 20.97, 22.37, and 22.72.

15. The crystalline form of any one of claims 11-13, wherein the crystalline form is characterized by X-ray powder diffraction (XRPD) peaks at values of two theta (°2θ±0.2°; or °2θ) of 3.61, 11.13, 11.70, 12.70, 16.25, 17.76, 19.09, 19.47, 20.29, 20.97, 22.37, and 22.72.

16. The crystalline form of claim 1 or 2, wherein the crystalline form (Form II) is characterized by at least three X-ray powder diffraction (XRPD) peaks at values of two theta (°2θ±0.2°; or °2θ) selected from 17.76, 19.47, 20.29, 20.97, and 22.37.

17. The crystalline form of claim 16, wherein the crystalline form is characterized by at least four X-ray powder diffraction (XRPD) peaks at values of two theta (°2θ±0.2°; or °2θ) selected from 17.76, 19.47, 20.29, 20.97, and 22.37.

18. The crystalline form of claim 16 or 17, wherein the crystalline form is characterized by at least five X-ray powder diffraction (XRPD) peaks at values of two theta (°2θ±0.2°; or °2θ) selected from 17.76, 19.47, 20.29, 20.97, and 22.37.

19. The crystalline form of any one of claims 16-18, wherein the crystalline form is characterized by X-ray powder diffraction (XRPD) peaks at values of two theta (°2θ±0.2°; or °2θ) of 17.76, 19.47, 20.29, 20.97, and 22.37.

20. The crystalline form of any of claims 11-19, wherein the crystalline form is characterized by an X-ray powder diffraction (XRPD) pattern substantially in accordance with that depicted in FIG. 4.

21. The crystalline form of any one of claims 11-20, wherein the crystalline form is further characterized by a DSC thermogram comprising a endothermic peak at about 160.4° C.

22. The crystalline form of any one of claims 11-21, wherein the crystalline form is further characterized by a DSC thermogram substantially in accordance with the DSC thermogram in FIG. 6.

23. The crystalline form of any one of claims 11-22, wherein the crystalline form is further characterized by a TGA thermogram substantially in accordance with the TGA thermogram in FIG. 6.

24. A method of treating or preventing a disease or disorder associated with a genetic defect in phenylalanine hydroxylase, comprising administering to a patient in need thereof an amount of the crystalline form of any one of claims 1-23.

25. The method of claim 24, wherein the compound reduces phenylalanine levels in the blood.

26. The method of claim 24 or 25, wherein the disease or disorder associated with a genetic defect in phenylalanine hydroxylase is phenylketonuria.

27. The method of claim 24 or 25, wherein the disease or disorder associated with a genetic defect in phenylalanine hydroxylase is hyperphenylalaninemia.

28. The method of any one of claims 24-27, wherein the compound inhibits SLC6A19 in the subject.
